(19) United States
(12) Patent Application Publication
(10) Pub. No.: US 2019/0231705 A1
(43) Pub. Date: Aug. 1, 2019

(54) HIGH DRUG LOAD POLYMERIC NANOPARTICLE FORMULATIONS AND METHODS OF MAKING AND USING SAME

(71) Applicant: Merck Sharp & Dohme Corp., Rahway, NJ (US)

(72) Inventors: Joseph Della Rocca, Blue Bell, PA (US); Angela M. Wagner, Austin, TX (US); Wei Xu, North Wales, PA (US); W. Peter Wuelfing, Harleysville, PA (US)

(73) Assignee: Merck Sharp & Dohme Corp., Rahway, NJ (US)

(21) Appl. No.: 16/255,333

(22) Filed: Jan. 23, 2019

Related U.S. Application Data
(60) Provisional application No. 62/623,099, filed on Jan. 29, 2018.

(51) Int. Cl. A61K 9/51; A61K 31/4985

(52) U.S. Cl. CPC A61K 9/5161; A61K 9/5138; A61K 9/5192; A61K 31/4985

(57) ABSTRACT

Described herein are polymeric nanoparticles that comprise {[(1R,25)-1-({[(1aR,5S,8S,10R,22aR)-5-tert-butyl-14-methoxy-3,6-dioxo-1,1a,3,4,5,6,9,10,18,19,20,21,22,22a-tetradecahydro-8H-7,10-methanocyclopropa[18,19][1,10,3,6]-dioxadiazacyclo-nonadecino[11,12-b]quinoxalin-8-yl]carbonyl}amino)-2-vinylcylopropyl]carbonyl}(cyclopropylsulfonyl)azanide, (i.e., grazoprevir), or a pharmaceutically acceptable salt thereof, and methods of making and using such nanoparticles.

### BACKGROUND OF THE INVENTION

One of the most critical missions of the drug delivery scientist is to effectively mitigate solubility limited absorption. Recent reviews have reported that up to 90% of all new drug candidate compounds can be classified as either BCS (Biopharmceutics Classification System) class II or IV. A number of different formulation strategies can be used to improve oral bioavailbility, including use of cosolvents/surfactants, cyclodextrin complexation, and particle size reduction. Solid dispersion formulations have been used previously to promote the oral absorption of poorly water soluble active pharmaceutical ingredients (APIs) (see Ford, 61 PHARM. ACTA HELV. 69-81 (1986)) and to minimize the effect of achlorhydria for weak bases (see M. A. Alam et al., 9(11) EXPERT OPIN. DRUG DELIVERY 1419-1440 (2012); A. Mitra et al., 8 MOL. PHARM. 2216-2223 (2011)). See also WO2016/012906, and M. Morgen et al., Pharm. Res. 2012 February:29(2): 427-40 29. Amorphous solid dispersions (ASD) have become one of the most powerful tools for maximing the oral bioavailability of compounds. Upon dissolution, ASD formulations can reach higher solubilities than the crystalline drug. A subset of ASD can disintegrate into drug-rich nanoparticles upon introduction in aqueous media. These ASD can generate in situ nanoparticles by either liquid-liquid phase separation or hydrophobic capture. Nano-forming ASD are believed to generate optimum oral bioavailibilty by maximizing the drug solubility and dissolution rate and may also diffuse into the unstirred water layer to provide higher local drug concentrations at the intenstinal epithelia. Unfortunately, in situ nanoparticle formation from an ASD has several limitations preventing it from being a universal formulation strategy. First, this approach has only been observed using poorly soluble, highly lipophillic API's. The dispersion itself frequently requires high levels of surfactants within the ASD and only works at low drug load. The design of in situ nanoparticle forming amorphous dispersions is highly empirical and time consuming.

Purposefully designed crystalline nanoparticles are well precedented in the literature and in several commerical products. Generally, an aqueous nanosuspension is created by either top down or bottom up methods, is dried into a solid, and then converted into a tablet or capsule. Rationally designed amorphous nanoparticle formulations have been more recently reported in the literature. Morgen and coworkers have reported an emulsion based approach to make ethylcellulose nanoparticles for several routes of administration. See Morgen, M.; Bloom, C.; Beyerinck, R.; Bello, A.; Song, W.; Wilkinson, K.; Steenwyk, R.; Shamblin, S., Polymeric Nanoparticles for Increased Oral Bioavailability and Rapid Absorption Using Celecoxib as a Model of a Low-Solubility, High-Permeability Drug. Pharmaceutical Research 29, (2), 427-440 and Morgen, M.; Lu, G. W.; Du, D.; Stehle, R.; Lembke, F.; Cervantes, J.; Ciotti, S.; Haskell, R.; Smithey, D.; Haley, K.; Fan, C. L., Targeted delivery of a poorly water-soluble compound to hair follicles using polymeric nanoparticle suspensions. International Journal of Pharmaceutics 416, (1), 314-322. Celecoxib/ethylcellulose nanoparticles provided a shorter Tmax and higher Cmax than a non-nanoparticle forming spray dried dispersion in a clinical study. Multiple groups have reported making amorphous nanoparticles by precipitation methods, with the most advanced reported formulations achieving single dose clinical studies.

Common approaches for yielding a high fraction of nanoparticles include creating amorphous intermediates by hot-melt extrustion (HME) or spray-drying (SD). However, designing HME or SD formulations that consistently yield a high fraction of nanoparticles during dissolution has proven to be challenging. A formulation platform that is geared specifically to generate high drug load amorphous nanoparticles could therefore have a significant impact on formulation of poorly-soluble compounds in general and on amorphous dispersion design in particular.

Grazopravir, (1aR,5S,8S, 10R,22aR)-N-[(1R,2S)-1-[(cyclopropylsulfonamido)-carbonyl]-2-ethenylcyclopropyl]-14-methoxy-5-(2-methylpropan-2-yl)-3,6-dioxo-1,1a,3,4,5,6,9,10,18,-19,20,21,22,22a-tetradecahydro-8H-7,10-methanocyclopropa[18,19][1,10,3,6]dioxadiaza-cyclononadecino[11,12-b]quinoxaline-8-carboxamide) is a hepatitis C virus protease inhibitor which acts at the NS3/4/a protease target, and is present in the drug ZEPATIER, which is manufactured by Merck Sharp & Dohme. It is a poorly water soluble, moderately lipophilic compound. These properties make conventional formulation approaches challenging.

Grazoprevir has the structure of formula I or a pharmaceutically acceptable salt thereof:

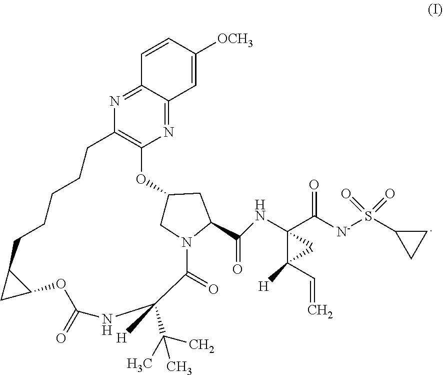 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]

Formula I is described in U.S. Pat. No. 7,973,040, incorporated by reference herein in its entirety. The SD formulation generates nanoparticles of grazoprevir upon dissolution within the GI tract. This invention discloses nanoparticle fromulations of grazoprevir at drug loads above those achievable by the commercial SD process. The nanoparticle formulations of this invention afford a higher drug load than the commercial formulation without sacrificing bio performance, with the ultimate implication being a smaller final product image, e.g., smaller tablet size.

### SUMMARY OF THE INVENTION

Described herein are stabilized polymeric nanoparticles, comprising a higher drug load of grazoprevir than the commercial formulation without sacrificing bio performance. In an embodiment, the polymeric nanoparticles comprise grazoprevir, or a pharmaceutically acceptable salt thereof (hereinafter referred to as, “Formula I”) and an anionic surface modifying agent. Further described, are methods of making and using such nanoparticles.

In one embodiment, polymer-stabilized nanoparticles are provided. The nanoparticles comprise from 0 to 15, 2 to 12, 4 to 10, 6 to 10, or 8 to 10 weight percent of an anionic surface modifying agent selected from sodium lauryl sulfate and sodium taurocholate, from 20 to 80, 25 to 70, or 30 to 65 weight percent of a water soluble polymer or poorly aqueous soluble polymer and from 20 to 80, 25 to 70, 30 to 68, or 33 to 66 weight percent of grazaprevir or a pharmaceutically acceptable salt thereof.

An embodiment of the invention is realized when the nanoparticle releases from 40 to 100% of grazoprevir when placed in solution at a temperature of about 35° C. to 37° C. An aspect of this invention is realized when the solution is intestinal fluid. Another aspect of this invention is realized when the solution is a fasted simulated intestinal solution at a temperature of about 35° C. to 37° C.

In another embodiment, the composition comprises nanoparticles that have an average size of less than 1000 nm.

An embodiment of this invention is realized when the released nanoparticles are about 100 to 500 or from 100 to 200 nm particles of pure amorphous Formula I. An embodiment of this invention is realized when the released nanoparticles are about 100 to 500 or about 100 to 200 nm particles of pure Formula I drug particle.

An embodiment of the invention is a suspension comprising the polymeric nanoparticles of grazoprevir. A subembodiment of this aspect of the invention is realized when the polymeric nanoparticles of grazoprevir in the suspension are stabilized and solidified within a redispersion matrix by spray drying. Another subembodiment of this aspect of the invention is realized when said redispersion matrix consists of a polymer matrix comprising a water soluble polymer or poorly aqueous soluble polymer, grazoprevir and anionic surface modifying agent. Another subembodiment of this aspect of the invention is realized when said redispersion matrix consists of a polymer matrix comprising a water soluble polymer or poorly aqueous soluble polymer, grazoprevir, anionic surface modifying agent, and one or more additives. Another subembodiment of this aspect of the invention is realized when the polymer matrix particle sizes are between 1-50 μm, 2-40 μm, 5-30 μm, 5-25 μm, or 5-20 μm with a drug load of about 20% to 80%, 25% to 70%, 30% to 70%, or 45% to 70%. An aspect of this invention is realized when the solidified nanoparticle matrix completely re-disperses grazoprevir nanoparticles within 1 to 15, 1 to 10, or 1 to 5 minutes of introduction into aqueous media conditions, for example intestinal fluid. An embodiment of this aspect of the invention is realized when the intestinal fluid is in a fasted state. Another embodiment of this aspect of the invention is realized when the temperature of the aqueous media condition is at about 37° C.

In another embodiment, the active agent in said nanoparticles is substantially non-crystalline.

In another embodiment, the composition comprises a water soluble polymer or poorly aqueous soluble polymer, grazoprevir, and anionic surface modifying agent.

In another embodiment, the composition comprising a water soluble polymer or poorly aqueous soluble polymer, grazoprevir, and anionic surface modifying agent collectively constitute at least 50 wt % of the nanoparticles.

An embodiment of this invention is realized when the surface modifying agent is sodium lauryl sulfate.

In another embodiment, grazoprevir is capable of forming a salt with counter-ions present in the nanosuspension media.

An embodiment of this invention is realized when the poorly water soluble polymer is converted into a salt form.

In any or all of these embodiments, the salt form in pure form may be substantially non-crystalline.

In embodiments of the invention, the polymer, grazoprevir and anionic surface modifying agent constitute an insoluble polymer matrix. In embodiments of the invention the polymer matrix may comprise additional additives.

### BRIEF DESCRIPTION OF THE DRAWINGS

FIG. 1: Dissolution Data of two different drug loads of grazoprevir/ethylcellulose nanoparticle suspensions compared to the Final Market Formulation (FMF) benchmark suspension.

FIG. 2: Dissolution of 50% DL grazoprevir/HPMCAS-H nanosuspension formulation (squares) and same formulation after spray drying (diamonds).

FIG. 3: Dissolution data of grazoprevir FMF formulation (triangle), 50% grazoprevir/HPMCAS-L nanoparticle suspension (square) and 50% grazoprevir/HPMCAS-L nanoparticles post spray drying (diamond).

FIG. 4: Dissolution of 45% DL grazoprevir/45% HPMCAS-L/10% addative nanoparticle formulations.

FIG. 5: Dissolution of grazoprevir/HPMCAS-L nanoformulations with different levels of SLS.

### DETAILED DESCRIPTION

Described herein are stabilized polymeric nanoparticles that comprise an active agent, preferably an NS3/4A inhibitor, and more preferably Hepatitus C agent, {[(1R,2S)-1-({[(1aR,5S,8S,10R,22aR)-5-tert-butyl-14-methoxy-3,6-dioxo-1,1a,3,4,5,6,9,10,18,19,20,21,22,22a-tetradecahydro-8H-7,10-methanocyclopropa[18,19][1,10,3,6]-dioxadiazacyclo-nonadecino[11,12-b]quinoxalin-8-yl]carbonyl }amino)-2-vinylcylopropyl]carbonyl (cyclopropylsulfonyl)azanide, (i.e., Formula I), or a pharmaceutically acceptable salt or solvate thereof, and methods of making and using such nanoparticles. In some embodiments, inclusion of an anionic surface modifying agent or surfactant in a disclosed nanoparticle and/or included in a nanoparticle preparation process may result in nanoparticles with improved drug loading. Furthermore, in certain embodiments, nanoparticles that include and/or are prepared in the presence of the anionic surfactant may exhibit improved release properties.

Various embodiments are disclosed herein. The description here is exemplary of the invention and is not intended to limit the scope, applicability, or configuration in any way. Various changes to the described embodiments may be made in the function and arrangement of the elements described herein without departing from the scope of the invention.

As used herein, the singular forms “a”, “an”, and “the” include the plural forms unless the context clearly indicates otherwise. Additionally, the term “includes” means “comprises.” Further, the term “coupled” generally means electrically, electronmagnectically, and/or physically (e.g., mechanically or chemically) coupled or linked and does not exclude the presence of intermediate elements between the coupled or associated items absent specific contrary language.

Unless otherwise indicated, all numbers expressing quantities of ingredients, properties such as molecular weight, percentages, and so forth, as used in the specification or claims are to be understood as being modified by the term “about.” Accordingly, unless otherwise indicated, the numberical parameters set forth are approximations that may depend on the desired properties sought and/or limits of detection under standard test conditions/methods.

As used herein “pKa” is defined as the negative logarithm of the ionization constant (K) of an acid, which is the pH of a solution in which half of the acid molecules are ionized.

The compositions disclosed herein may be suitable for use with any ionizable biologically active compound desired to be administered to a patient in need thereof. The compositions may contain one or more active agents. As used herein, by “active” or “active agent” is mean a drug, medicament, pharmaceutical, therapeutic agent, nutraceutical, or other compound that may be desired to be administered to the body. The active is generally a “small molecule” having a molecular weight of 2000 Daltons or less. Grazoprevir is a preferred active agent.

By “ionizable active agent” is meant an agent that is ionizable in the physiolocial pH range of the gastro-intestinal tract, such as pH from 1 to 8. An active agent as used herein is an active agent that is essentially totally water-insoluble or poorly water-soluble at any pH in the range of pH 2.0 to pH 8.0. In one embodiment, the active agent has a water solubility of less than 5 mg/mL at any pH in the range of pH 2.0 to pH 8.0. The active agent may have an even lower water solubility at any pH in the range of pH 2.0 to pH8.0 such as less than 1 mg/mL, and less than 0.1 mg/mL.

The active agent should be understood to include the non-ionized and ionized form of the active. By non-ionzed form it is meant that the active is not converted to an ionic form. By ionized form it is meant that in at least one pH in the physiolocial pH range of 1 to 8, the active is converted to an ionic form, i.e., a cation or anion. In one embodiment, the ionizable active agent includes pharmaceutically acceptable forms of the active. The term “pharmaceutically acceptable” refers to a substance that can be taken into a subject without significant adverse toxicological effects on the subject. By “pharmaceutically acceptable forms” it is meant any pharmaceutically acceptable derivative or variation, including salts, stereoisomers, stereoisomer mixtures, enantiomers, solvates, hydrates, isomorphs, polymorphs, pseudomorphs, neutral forms and prodrugs of the active agent.

Examples of NS3/4A protease inhbitors useful in this invention are selected from Asunaprevir, Boceprevir, Ciluprevir, Danoprevir, Faldaprevir, Glecaprevir, Grazoprevir, Narlaprevir, Paritaprevir, Simeprevir, Sovaprevir, Telaprevir, Vaniprevir, Vedroprevir, Voxilaprevir); NSSA inhibitors (Daclatasir, Elbasvir (described in U.S. Pat. No. 8,871,759), Ledipasvir, Odalasvir, Ombitasvir, Pibrentasvir, Ravidasvir, Ruzasvir, Samatasvir, Velpatasvir). Also useful in this invention are NSSB RNA polymerase inhibitors such as Beclabuvir, Dasabuvir, Deleobuvir, Filibuvir, Setrobuvir, Sofosbuvir, Radalbuvir, and Uprifosbuvir.

In an embodiment the ionizable group on the active agent is anionic. In another embodiment, the ionizable group is anionic and selected from carboxylic acids, acetates, including trifluoroacetate salts, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

In embodiments of the invention, the surface modifying agent or surfactant is anionic and is selected from the group consisting of sodium lauryl sulfate, sodium glyocholate, sodium taurocholate, or a mixture thereof An aspect of this embodiment is realized when the anionic surface modifying agent is sodium lauryl sulfate. Another aspect of this embodiment is realized when the anionic surface modifying agent is sodium glycolate.

In any or all of the above embodiments, the anionic surface modifying agent may be from about 0.5 to 25% on a weight basis to the active agent. A sub-embodiment of this aspect of the invention is realized when the anionic surface modifier may be about 10 to 25% on a weight basis to the active agent. A sub-embodiment of this aspect of the invention is realized when the anionic surface modifier may be about 15 to 25% on a weight basis to the active agent. A sub-embodiment of this aspect of the invention is realized when the anionic surface modifier may be about 20 to 24% on a weight basis to the active agent. Another sub-embodiment of this aspect of the invention is realized when the anionic surface modifier may be about 22% on a weight basis to the active agent.

Embodiments of the disclosed compositions when prepared by emulsification or precipitation comprise at least one poorly aqueous soluble polymer. As used herein, the term “poorly aqueous soluble” means a polymer that has a solubility of less than 10 mg/ml when administered alone at a concentration of from 50 to 100 mg/ml in water. A test to determine whether the polymer is poorly aqueous may be performed as follows. The polymer is initially present in bulk powder form with average particle sizes of greater than 1 micron. The polymer alone is administered to the buffer solution and stirred for approximately 1 hour at room temperature. The polymer suspension is then filtered through a PDVF (0.45 um) and the polymer concentration in the filtrate is determined by liquid chromatography/charged aerosol detection (LC-CAD) under several different dilutions. The polymer is considered to be poorly aqueous soluble if it has a solubility of less than 10 mg/ml in this test. In some embodiments, polymers for use in the invention have a solubility of less than 5 mg/ml in this test, less than 1 mg/ml in this test, or even less than 0.1 mg/ml in this test.

In some embodiments, the poorly aqueous soluble polymer is selected from the group consisting of cellulose, ethylcellulose (EC), propylcellulose, butylcellulose, cellulose acetate (CA), cellulose propionate, cellulose butyrate, cellulose acetate butyrate (CAB), cellulose acetate propionate, methyl cellulose acetate, methyl cellulose propionate, methyl cellulose butyrate, ethyl cellulose acetate, ethyl cellulose propionate, ethyl cellulose butyrate, hydroxypropyl methylcellulose acetate (HPMCA), dextran acetate, dextran propionate, dextran acetate propionate, polyvinyl caprolactam, polyvinyl acetate, polyoxyethylene castor oils, polycaprolactam, polylactic acid, polyglycolic acid, polylactic-glycolic)acid, hydroxypropyl methyl cellulose acetate succinate (HPMCAS), hydroxypropyl methyl cellulose propionate succinate, hydroxypropyl methyl cellulose phthalate (I-IPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose acetate phthalate (HPMCAP), hydroxypropyl methylcellulose propionate phthalate, hydroxypropyl methylcellulose acetate trimellitate (HPMCAT), carboxymethyl cellulose acetate butyrate (CMCAB), hydroxypropyl methylcellulose propionate trimellitate, cellulose acetate succinate (CAS), methyl cellulose acetate succinate (MCAS), dextran acetate succinate, dextran propionate succinate, dextran acetate propionate succinate, poly(methacrylic acid-co-methyl methacrylate) 1:1 (e,g., Eudragit® L100, Evonik Industries AG), poly(methacrylic acid-co-methyl methacrylate) 1:2 (e.g., Eudragit® 8100), poly(methacrylic acid-co-ethyl acrylate) 1:1 (e.g., Eudragit® L100-55), poloxamers, poly(ethylene oxide-b- [symbol] -caprolactone caprolactone), poly(E-caprolactone-b-ethylene glycol), poly(ethylene oxide-b-lactide), poly(lactide-b-ethylene glycol), poly(ethylene oxide-b-glycolide), poly(glycolide-b-ethylene glycol), poly(ethylene oxide-b-lactide-co-glycolide), poly(lactide-co-glycolide-b-ethylene glycol), and mixtures thereof. A sub-embodiment of this aspect of the invention is realized when the poorly aqueous soluble polymer is selected from the group consisting of hydroxypropylmethylcellulose (HPMCAS), ethylcellulose (EC), propylcellulose, and butylcellulose. A sub-embodiment of this aspect of the invention is realized when the poorly aqueous soluble polymer is hydroxypropylmethylcellulose (HPMCAS). A sub-embodiment of this aspect of the invention is realized when the poorly aqueous soluble polymer is ethylcellulose (EC).

In another embodiment, the poorly aqueous soluble polymer has a pH-dependent solubility profile. In one embodiment, the poorly aqueous soluble polymer is an enteric polymer, having a low aqueous solubility at pH levels below 6. Exemplary enteric polymers include hydroxypropyl methyl cellulose acetate succinate (HPMCAS), hydroxypropyl methyl cellulose, propionate succinate, hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose acetate phthalate (HPMCAP), hydroxypropyl methylcellulose propionate phthalate, hydroxypropyl methylcellulose acetate trimellitate (HPMCAT), carboxymethyl cellulose acetate butyrate (CMCAB), hydroxypropyl methylcellulose propionate trimellitate, cellulose acetate succinate (CAS), methyl cellulose acetate succinate (MCAS), dextran acetate succinate, dextran propionate succinate, dextran acetate propionate succinate, poly(methacrylic acid-co-methyl methacrylate) 1:1 (e.g., Eudragit® L100, Evonik Industries AG), poly(methacrylic acid-co-methyl methacrylate) 1:2 (e.g., Eudragit® 8100), poly(methacrylic acid-co-ethyl acrylate) 1:1 (e.g., Eudragit® L100-55), and mixtures thereof.

In another embodiment, the poorly aqueous soluble polymer has a pH-dependent solubility profile. In this embodiment, the polymer is poorly soluble under a range under a subset of the physiologically relevant pH range, but can be made made water soluble by titrating the pH above a certain threshold value. A sub-embodiment of this invention is realized when the poorly aqueous soluble polymer is HPMCAS. At the target concentrations of the precipitation method, the polymer has poor aqueous solubility when suspended in water, but becomes fully soluble once the pH is raised above 6.

In another embodiment, the poorly aqueous soluble polymer is capable of forming micelles at a low concentration in water. By “low concentration” is meant a concentration of less than 5 wt % in water, such as a concentration of less than 4 wt %, less than 3 wt %, or less than 2 wt %. By “micelles” is meant an aggregate of molecules present as a colloid or nanoparticle of less than 100 nm in diameter. When poorly aqueous soluble polymers are added to an aqueous solution, there is typically a concentration above which the polymer molecules aggregate. These aggregates can be called micelles or nanoparticles, and they can be analyzed by light scattering analysis, such as dynamic light scattering (DLS) techniques, turbidity measurements, or imaging techniques such as electron microscopy. Exemplary poorly aqueous soluble polymers that form micelles include poloxamers, poly(ethylene oxide-b- [symbol] -caprolactone), poly( [symbol] -caprolactone-b-ethylene glycol), poly(ethylene oxide-b-lactide), poly(lactide-b-ethylene glycol), poly(ethylene oxide-b-glycolide), poly(glycolide-b-ethylene glycol), poly(ethylene oxide-b-lactide-co-glycolide), poly(lactide-co-glycolide-b-ethylene glycol), and mixtures thereof.

Embodiments of the disclosed compositions, when prepared by precipitation, optionally comprise at least one water soluble polymer. In some embodiments, the water soluble polymer is selected from the group consisting polyvinylpyrollidone-vinyl alcohol (PVP-V A64), polyvinyl pyrollidinone, polyethylene glycol, and polyvinyl alcohol, or a mixture thereof. A sub-embodiment of this aspect of the invention is realized when the water soluble polymer is polyvinylpyrollidone-vinyl alcohol (PVP-V A64). Another sub-embodiment of this aspect of the invention is realized when the water soluble polymer is polyvinyl pyrollidinone. Another sub-embodiment of this aspect of the invention is realized when the water soluble polymer is polyethylene glycol. Another sub-embodiment of this aspect of the invention is realized when the water soluble polymer is polyvinyl alcohol.

In any or all of some embodiments, the composition comprises nanoparticles comprising a water soluble polymer or poorly aqueous soluble polymer, an NS3 inhibitor, or a pharmaceutically acceptable salt or solvate thereof, an anionic surface modifying agent, and optionally an additive selected from the group consisting of sucrose, lactose, mannitol, casein, trehalose, cellulose and mesoporous silica, or a mixture thereof. An embodiment of this invention is realized when the surface modifying agent is sodium lauryl sulfate or sodium taurocholate. An embodiment of this invention is realized when the water soluble polymer or poorly aqueous soluble polymer, anionic surface modifying agent and optional additive constitute a polymer matrix. A subembodiment is realized when the range of water soluble polymer or poorly aqueous soluble polymer ranges from 20-80%, 25-65%, or 33-60%, the surface modifiying agent ranges from 0.5-15%, or 5-10%, the additive ranges from 0-10% or 0.1-10%, and the NS3 inhibitor ranges from 25-80%. A sub-embodiment of this aspect of the invention is realized when the additive is sucrose. Another sub-embodiment of this aspect of the invention is realized when the additive is lactose. Another sub-embodiment of this aspect of the invention is realized when the additive is mannitol. Still another sub-embodiment of this aspect of the invention is realized when the additive is casein. Still another sub-embodiment of this aspect of the invention is realized when the additive is trehalose. Yet another sub-embodiment of this aspect of the invention is realized when the additive is cellulose. Another sub-embodiment of this aspect of the invention is realized when the additive is mesoporous silica.

In any or all of the above embodiments, the additive may be about 0 to about 15 percent on a weight basis to the active agent.

In any or all of the above embodiments, the molar ratio of Compound A to the anionic surface modifying agent or surfactant may be at least 1.0. In some embodiments, the molar ratio of Compound A to the surfactant is greater than 1.0.

In any or all of the above embodiments, the nanoparticles may have an average size of less than 500 nm, less than 400 nm, less than 300 nm, or less than 200 nm.

In some embodiments, the disclosed compositions are made by an emulsification process which includes 1) forming an organic solution comprising an organic solvent, Compound A, and a poorly aqueous soluble polymer; 2) forming an aqueous solution with the surface modifying agent, wherein Compound A, and poorly aqueous soluble polymer are poorly soluble in said aqueous solution; 3) mixing said organic solution with said aqueous solution to form a first mixture; and 4) removing at least a portion of said organic solvent from said first mixture to form a suspension comprising the said nanoparticles and said aqueous solution, wherein said nanoparticles have a size of less than 1000 nm, wherein Compound A, the surface modifying agent, and the poorly aqueous soluble polymer collectively constitute at least 60 wt % of said nanoparticles. In some embodiments, the organic solvent is removed by spray drying, evaporation, extraction, diafiltration, pervaporation, vapor permeation, distillation, filtration, or any combination thereof.

In some embodiments, the organic solvent in the emulsification process is immiscible with the aqueous solution. In an embodiment, the organic solvent is methylene chloride, trichloroethylene, tetrachloroethane, trichlorethane, dichloroethane, dibromoethane, ethyl acetate, benzyl alcohol, phenol, chloroform, toluene, xylene, ethyl-benzene, methyl-ethyl ketone, methyl-iosbutyl ketone, or any mixture thereof. In another embodiment, the organic solvent is methylene chloride, ethyl acetate, benzyl alcohol, or any mixture thereof.

In some embodiments, the process is a precipitation process, and the organic solvent is miscible with the aqueous solution. In one embodiment, the organic solvent is acetone, methanol, ethanol, tetrahydrofuran (THF), dimethylsulfoxide (DMSO), or any mixture thereof. In an aspect of this embodiment, Compound A can be ionized by pH modifying agents selected from sodium hydroxide, potassium hydroxide, ammonia, sodium phosphate, lysine and arginine. In some embodiments, the disclosed compositions are made by a precipitation process which includes 1) forming an organic solution comprising an organic solvent and the active agent, 2) forming an aqueous solution composed of the dissolved polymer and optionally an anionic surface modifying agent, 3) mixing said solutions together to form a suspension which forms nanoparticles having a diameter of less than 1000 nm and 4) removing said solvents from the suspension to form a heterogeneous micron sized particle of the said nanoparticles embedded within the polymer and surface modifying agent. In some embodiments, the solvents are removed by spray drying, evaporation, extraction, diafiltration, pervaporation, vapor permeation, distillation, filtration, or any combination thereof. In other embodiments, the polymer can be dissolved in the organic solution with the active agent.

The disclosed compositions may be formulated into a dosage form. In some embodiments, the dosage form can be tablets, capsules, suspensions, powders for suspension, creams, transdermal patches, depots, or any combination thereof In some embodiments, the dosage form is formulated for administration to an animal via oral, buccal, mucosal, sublingual, intravaenous, intra-arterial, intramuscular, subcutaneous, intraperitoneal, intraarticular, infusion, intrathecal, intraurethral, topical, subdermal, transdermal, intranasal, inhalation, pulmonary tract, intrtracheal, intraocular, ocular, intraaural, vaginal, or rectal delivery. In one embodiment, the dosage form is formulated for intravenous, intra-arterial, intramuscular, subcutaneous, intraperitoneal, intraarticular, infusion, intrathecal, or intraurethral delivery. In another embodiment, the dosage form is formulated for topical administration, and the formulation is a gel, hydrogel, lotion, liposome, solution, cream, ointment, foam, bandage or microemulsion.

Embodiments of the disclosed compositions comprise nanoparticles comprising a water soluble polymer or poorly aqueous soluble polymer, an ionizable active agent that is Compound A, and an anionic surface modifying agent having an opposite charge as the ionizable active agent. In one embodiment, the poorly aqueous soluble polymer, the ionizable active agent, and the anionic surface modifying agent having an opposite charge as the ionizable active agent collectively constitute at least 50 wt % of the nanoparticles. In another embodiment, the water soluble polymer, or poorly aqueous soluble polymer, an ionizable active to agent, and an anionic surface modifying agent having an opposite charge as the ionizable active agent collectively constitute at least 80 wt % of the nanoparticles. In still another embodiment, the water soluble polymer or poorly aqueous soluble polymer, an ionizable active agent, and an anionic surface modifying agent having an opposite charge as the ionizable active agent collectively constitute at least 90 wt % of the nanoparticles. In another embodiment, the composition consists essentially of the water soluble polymer or poorly aqueous soluble polymer, the ionizable active agent, and the anionic surface modifying agent having an opposite charge as the ionizable active agent.

As used herein, “consists essentially of” means that the composition may include small amounts (i.e., less than 5 wt %) of other components that do not materially alter the composition, such as other excipients.

In another embodiment, the composition comprises water soluble polymer, or poorly aqueous soluble polymer, the ionizable active agent, and the anionic surface modifying agent having an opposite charge as the ionizable active agent. An embodiment of this invention is realized when the surface modifying agent is sodium lauryl sulfate or sodium taurocholate. The nanoparticles are small particles comprising a water soluble or poorly aqueous soluble polymer, an ionizable active agent that is Compound A, and an anionic surface modifying agent. An embodiment of this invention is realized when the surface modifying agent is sodium lauryl sulfate or sodium taurocholate. By “nanoparticles” is meant a plurality of small particles in which the average size of the particles is less than 1000 nm. In suspension, by “size” is meant the effective cumulant diameter as measured by dynamic light scattering (DLS), using for example, Brookhaven instruments’ 90Plus particle sizing instrument. By “size” is meant the diameter if the particles were spherical particles. In one embodiment, the average size of the nanoparticles is less than 500 nm, less than 400 nm, less than 300 nm, less than 200 nm, less than 150 nm, or even less than 100 nm.

In one embodiment, the nanoparticles of the invention consist essentially of a poorly aqueous soluble polymer, an ionizable active agent, and an anionic surface modifying agent. In other embodiments, the nanoparticles of the invention consist of a poorly aqueous soluble polymer, an ionizable active agent, and an anionic surface modifying agent.

In one embodiment, the nanoparticles of the invention consist essentially of a water soluble polymer, an ionizable active agent, and an anionic surface modifying agent. In other embodiments, the nanoparticles of the invention consist of a water soluble polymer, an ionizable active agent, and an anionic surface modifying agent.

In one embodiment, the amount of ionizable active agent in the nanoparticles may range from 0.01 wt % to 99 wt % active. In other embodiments, the amount of active may range from 0.1 wt % to 80 wt %, or from 0.1 to 60 wt %, or from 1 to 40 wt %. In other embodiments, the amount of active in the nanoparticles may be at least 1 wt %, at least 5 wt %, at least 10 wt %, at least 15 wt %, at least 20 wt %, at least 25 wt %, at least 30 wt %, at least 40 wt %, at least 45 wt %, or even at least 50 wt %.

In one embodiment, the active agent, grazoprevir, is present in the nanoparticles as a salt form of the active and the anionic surface modifying agent.

In one embodiment, the active in the nanoparticles (or the salt form of the active and the surface modifying agent) is present in a non-crystalline state. In one embodiment, the non-crystalline active exists as a solid solution of pure active homogeneously distributed throughout the polymer, or any combination of these states or those that lie intermediate between them. A “solid solution” is formed when at least one solid component is molecularly dispersed within another solid component, resulting in a homogeneous or substantially homogeneous solid material. In another embodiment, at least 80 wt % of the active in the nanoparticles is present in a non-crystalline state. In another embodiment, at least 90 wt % of the active in the nanoparticles is present in a non-crystalline state. In still another embodiment, essentially all of the active in the nanoparticles is in a non-crystalline state. In one embodiment, the non-crystalline active is present as a solid solution of active homogeneously distributed throughout the polymer.

The amount of non-crystalline active in the nanoparticles may be measured by powder X-ray diffraction (PXRD) or modulated differential scanning calorimetry (mDSC), or any other standard quantitative measurement.

In one method of forming embodiments of the disclosed nanoparticles, the ionizable active agent, the anionic surface modifying agent and a poorly aqueous soluble polymer are mixed with a solvent to form a liquid solution or liquid suspension. The nanoparticles may then be formed from the liquid solution or suspension by any known process, including precipitation in a miscible non-solvent, emulsifying in an immiscible non-solvent, or by forming droplets followed by removal of the solvent by evaporation to produce particles.

The nanoparticles may be formed by any process that results in formation of nanoparticles of an ionizable active agent, an anionic surface modifying agent, and a water soluble, or poorly aqueous soluble polymer.

One process for forming nanoparticles is an emulsification process. In this process, the ionizable active agent, anionic surface modifying agent, and poorly aqueous soluble polymer are dissolved in an organic solvent that is immiscible with an aqueous solution in which the active agent, anionic surface modifying agent, and poorly aqueous soluble polymer are poorly soluble, forming an organic solution. Solvents suitable for forming the organic solution of dissolved active agent, counterion, and polymer can be any compound or mixture of compounds in which the active agent, anionic surface modifying agent, and the poorly aqueous soluble polymer are mutually soluble and which is immiscible with the aqueous solution. As used herein, the term “immiscible” means that the organic solvent has a solubility in the aqueous solution of less than 20 wt %. In sonic embodiments, the organic solvent has a solubility in the aqueous solution of less than 10 wt %, or less than 5 wt %, or even less than 3 wt %. In one embodiment, the organic solvent is also volatile with a boiling point of 150° C. or less. Exemplary solvents include methylene chloride, trichloroethylene, tetrachloroethane, trichloroethane, dichloroethane, dibromoethane, ethyl acetate, phenol, chloroform, toluene, xylene, ethyl-benzene, methyl-ethyl ketone, methyl-isobutyl ketone, and mixtures thereof. In one embodiment, the organic solvent is methylene chloride, ethyl acetate, benzyl alcohol, or a mixture thereof. In one embodiment, the aqueous solution is water.

Once the organic solution is formed, it is then mixed with the aqueous solution and mixed at high shear, for example via homogenization, to form an emulsion of fine droplets of the water immiscible solvent distributed throughout the aqueous phase. The volume ratio of organic solution to aqueous solution used in the process will generally range from 1:100 (organic solution:aqueous solution) to 2:3 (organic solution:aqueous solution). In one embodiment, the organic solution:aqueous solution volume ratio ranges from 1:9 to 1:2 (organic solution:aqueous solution). The emulsion is generally formed by a two-step homogenization procedure. The solution of active agent, anionic surface modifying agent, poorly aqueous soluble polymer and organic solvent are first mixed with the aqueous solution using a rotor/stator or similar mixer to create a “pre-emulsion”. This mixture is then further processed with a high-pressure homogenizer that subjects the droplets to very high shear, creating a uniform emulsion of very small droplets. A portion of the solvent is then removed such that the droplets solidify forming a suspension of the nanoparticles in the aqueous solution. Exemplary processes for removing the organic solvent include spray drying, evaporation, extraction, diafiltration, pervaporation, vapor permeation, distillation, filtration, and combinations thereof. In one embodiment, the solvent is removed to a level that is acceptable according to The International Committee on Harmonization (ICH) guidelines. In one embodiment, the concentration of solvent in the nanoparticle suspension is less than the solubility of the solvent in the aqueous solution. Even lower concentrations of solvent may be obtained. Thus, the concentration of solvent remaining in the nanoparticle suspension following the removal steps may be less than 5 wt %, less than 3 wt %, less 1 wt %, and even less than 0.1 wt %.

An alternative process to form the nanoparticles is a precipitation process. In this process, the active agent, anionic surface modifying agent, and water soluble polymers are first dissolved in an organic solvent that is miscible with an aqueous solution in which the active agent, anionic surface modifying agent, and water soluble polymers are poorly soluble to form an organic solution. The organic solution is mixed with the aqueous solution causing the nanoparticles to precipitate. Solvents suitable for forming the organic solution of dissolved active agent, anionic surface modifying agent, and water soluble polymers can be any compound or mixture of compounds in which the active agent, anionic surface modifying agent, and the water soluble polymer are mutually soluble and which is miscible with the aqueous solution. In one embodiment, the organic solvent is also volatile with a boiling point of 150° C. or less. In addition, the organic solvent should have relatively low toxicity. Exemplary solvents include acetone, methanol, ethanol, tetrahydrofuran (THF), dimethylsulfoxide (DMSO), and ethyl acetate (as long as the aqueous:organic volume ratio is greater than 15). Mixtures of solvents, such as 50% methanol and 50% acetone, can also be used, so long as the active agent, anionic surface modifying agent, and water soluble polymers, are sufficiently soluble to dissolve the active agent, anionic surface modifying agent, and water soluble polymers. In one embodiment, the solvent is methanol, acetone, or a mixture thereof. The aqueous solution may be any compound or mixture of compounds in which the active agent, anionic surface modifying agent, and water soluble polymers are sufficiently insoluble so as to precipitate to form nanoparticles. In one embodiment, the aqueous solution is water. The water may contain additives, such as acid, base, or buffers to control the pH, surfactants such as sodium glycocholate to control the interfacial tension or salts to control the ionic strength of the aqueous solution.

The organic solution and aqueous solution are combined under conditions that cause solids to precipitate as nanoparticles. The mixing can be by addition of a bolus or stream of organic solution to a stirring container of the aqueous solution. Alternately a stream or jet of organic solution can be mixed with a moving stream of aqueous solution. In either case, the precipitation results in the formation of a suspension of nanoparticles in the aqueous solution.

For the precipitation process, the amount of active agent, anionic surface modifying agent and water soluble polymers in the organic solution depends on the solubility of each in the organic solvent and the desired ratios of active agent and counterion to polymers in the resulting nanoparticles. The organic solution may comprise from 0.1 wt % to 20 wt % dissolved solids, such as a dissolved solids content of from 0.5 wt % to 10 wt %.

The organic solution:aqueous solution volume ratio should be selected such that there is sufficient aqueous solution in the nanoparticle suspension that the nanoparticles solidify and do not rapidly agglomerate. However, too much aqueous solution will result in a very dilute suspension of nanoparticles, which may require further processing to concentrate the nanoparticles for ultimate use. Generally, the organic solution:aqueous solution volume ratio is at least 1:100, but generally is less than 1:2 (organic solution:aqueous solution). In one embodiment, the organic solution:aqueous solution volume ratio ranges from 1:10 to 1:3.

Once the nanoparticle suspension is made, a portion of the organic solvent may be removed from the suspension using methods known in the art. Exemplary processes for removing the organic solvent include evaporation, spray drying, extraction, diafiltration, pervaporation, vapor permeation, distillation, filtration, and combinations thereof. In one embodiment, the solvent is removed to a level that is acceptable according to ICH guidelines. Thus, the concentration of solvent in the nanoparticle suspension may be less than 10 wt %, less than 5 wt %, less than 3 wt %, less than 1 wt %, and even less than 0.1 wt %.

Both the emulsion process and the precipitation process result in the formation of a suspension of the nanoparticles in the aqueous solution. In some instances it is desirable to concentrate the nanoparticles or to isolate the nanoparticles in solid form by removing some or all of the liquid from the suspension. Exemplary processes for removing at least a portion of the liquid include spray drying, spray coating, spray layering, lyophylization, evaporation, vacuum evaporation, filtration, ultrafiltration, reverse osmosis, and other processes known in the art. In one embodiment, the process is spray drying. In one embodiment, the process is lyophilization. One or more processes may be combined to remove the liquid from the nanoparticle suspension to yield a solid composition. For example, a portion of the liquids may be removed by filtration to concentrate the nanoparticles, followed by spray-drying to remove most of the remaining liquids, followed by a further drying step such as tray-drying. Alternatively, the liquids may be removed by lyophilization.

In certain embodiments, the disclosed nanoparticles are in the form of particles of a solid dry powder, substantially all particles comprising a plurality of nanoparticles. As used herein, the term “particles” means small pieces of matter having characteristic diameters of less than 3000 μm. In another embodiment, the particles are granulated into granules using standard methods known in the art, such as dry granulation, wet granulation, high shear granulation, and the like. In one embodiment, the mean size of the particles is less than 500 μm. In another embodiment, the mean size of the particles is less than 200 μm. In still another embodiment, the mean size of the particles is less than 100 μm. In one embodiment, the mean size of the particles ranges from 0.5 to 500 μm. In another embodiment, the mean size of the particles ranges from 0.5 to 200 μm. In one embodiment, the mean size of the particles ranges from 0.5 to 100 μm. In one embodiment, the mean size of the particles ranges from 10 to 100 ∞m. In one embodiment, the mean size of the particles ranges from 10 to 70 μm. In one embodiment, the mean size of the particles ranges from 10 to 50 μm. In one embodiment, the mean size of the particles ranges from 0.5 to 10 μm. In one embodiment, the mean size of the particles ranges from 0.5 to 7 μm.

In one embodiment, the salt form of the ionizable active agent with the anionic surface modifying agent having an opposite charge as the ionizable active agent may be made and isolated, followed by forming a composition as disclosed herein. In another embodiment, the salt form of the active and the counterion may be formed in situ in an appropriate solvent, and subsequently formed into a composition as disclosed herein.

When isolating the nanoparticles in solid form, it is often desirable to include a matrix material in the suspension of nanoparticles prior to removal of the liquids. The matrix material functions to help slow or prevent agglomeration of the nanoparticles as the liquids are being removed, as well as to help re-suspend the nanoparticles when the solid composition is added to an aqueous solution (e.g., an aqueous environment of use) In one embodiment, the matrix material is pharmaceutically acceptable and water soluble. Examples of matrix materials include polyvinyl pyrrolidone (PVP), trehalose, hydroxypropyl methyl cellulose (HPMC), hydroxypropyl cellulose (HPC), carboxymethyl ethylcellulose (CMEC), hydroxypropyl methylcellulose acetate succinate (HPMCAS), casein, caseinate, albumin, gelatin, acacia, sucrose, cellulose, mesoporous silica, lactose, mannitol, pharmaceutically acceptable forms thereof, and other matrix materials known in the art.

In another aspect, a pharmaceutically acceptable composition is provided. The pharmaceutically acceptable composition comprises a plurality of contemplated nanoparticles and a pharmaceutically acceptable excipient.

In yet another aspect, a method of treating Hepatitis C, or cancer (e.g., including, but not limited to, prostate cancer, breast cancer, and ovarian cancer) in a patient in need thereof is provided. The method comprises administering to the patient a therapeutically effective amount of a composition comprising nanoparticles contemplated herein.

### Methods of Manufacture

### EXAMPLES

The invention now being generally described, it will be more readily understood by reference to the following examples which are included merely for purposes of illustration of certain aspects and embodiments, and are not intended to limit the invention in any way.
General: All materials were purchased from Thermofisher Scientific unless explictly noted. grazoprevir and methods for making are described in U.S. Pat. No. 7,973,040. HPMCAS-L (obtained from Shin-Etsu) and SLS (obtained from Sigma Aldrich). Ethylcellulose (grade 4) was obtained from Dow Chemical Company. Dynamic Light Scattering: Nanoparticle suspensions were diluted into either water, fasted state simulated intestinal fluid (fassif), or SGF targeting a 0.5 mg/mL total solids concentration. Suspension particle size was measured using either a Malvern Zetasizer nano or a Wyatt DynaPro DLS.
Filter Potency: Size fractionization of the nanosuspensions were achieved by passing the nanosuspensions through either a 1 μm or 0.45 μm glass fiber filter. Compound A levels were quantified via UPLC.
Biorelevant dilution: Biorelevant dissolution was performed by placing the test formulation in SGF for 30 minutes before sample analysis. A portion is diluted into Fasted State Simulated Intestinal Fluid (Fassif) to a target concentration(50 μg/mL). Samples were taken at 10, 20, 30 and 60 minutes after the dilution step and assayed via RP-HPLC. All test formulations were compared to a Commerically marketed formulation for compound A. RP-HPLC was recorded on an Agilent 1100 HPLC with a binary pump and UV detection. The separation was carried out on a 5cm×4.6 mm ID column packed with a C18 Stationary Phase (Xbridge C18, Waters Corp.) using the aforementioned pump and UV detection at 340 nm. The mobile phase was composed of a 50:50 mix of 10 potassium phosphate, pH 7.5:Acetonitrile flowing at 2 mL/min at a column temperature of 45° C.

Grazoprevir/Ethylcellulose Based Formulations

Production of grazoprevir/ethylcellulose nanoparticles: Compound A/ethylcellulose nanoparticles were made by a modification of a literature method.^{1,2}

66% Drug Load (DL)Nanoparticle: Compound A (400 mg) and ethylcellulose (200 mg) were stirred into 9 mL of dichloromethane until all components were fully dissolved. An aqeuous solution (40 mL) of 1 mg/mL sodium lauryl sulfate (SLS) was prepared. The aqueous and organic solutions were mixed together using a high shear rotostator (Polytron PT10-35GT) at 11,000 rpm for 4 minutes. This course emulsion was loaded onto a Microfluidics M110L high pressure homogenizer outfitted with a H10Z (z-cell interaction chamber and a cooling cell connected to a Neslab RTE 30 chiller set to 5 C. The processing pressure of the instrument was set to 12,500 psi and emulsion was processed for 12 minutes (100 passes through the interaction chamber). The nanoemulsion was collected from the homogenizer and an additional 20 mL of aqueous solution was added. This solution was processed for 2 minutes to collect any residual material within the unit and combined with the original emulsion. Dichloromethane was removed from the system via rotary evaporation and residual solvent levels were quantified via gas chromatography. Particle size was characterized by DLS and suspension drug levels were quantified by UPLC. All suspensions were used within 36 hours of generation.

Characterization (DLS): Diameter: 132.8±2 nm; % PD: 13.4. (UPLC): grazoprevir: 6.8 mg/mL
33% Drug Load Nanoparticle: grazoprevir (400 mg) and ethylcellulose (800 mg) were stirred into 9 mL of dichloromethane until all components were fully dissolved. Otherwise, the production procedure was identical to the 66% DL case.

Characterization (DLS): Diameter: 115.8±2 nm; % PD: 12.6. UPLC: grazoprevir: 4.84 mg/M1

Dissolution Data of grazoprevir-ethylcellulose Nanoparticles

FIG. 1 describes dissolution data of two different grazoprevir/ethylcellulose nanoparticle formulations compared to the commerically marketed formulation. The commerical formulation achieved the highest extent of release, but all three formulations achieved their top value within 5 minutes of Fassif introduction.

40% Drug Load Nanoparticle: grazoprevir (40 mg) and ethylcellulose (60 mg) were dissolved into 6 mL of dichoromethane until all components were fully dissolved. This was combined with an aqueous solution of either 1 mg/mL sodium glycholate or 0.4 mg/mL SLS. The coarse emulison was prehomogenized on a high shear rotator for 3 minutes and the transferred to the M110L high pressure homogenizer. The suspension was homogenized for 6 minutes (˜100 passes) and the emulsion was collected. Dichloromethane was removed by rotory evaporation. The nanosuspensions were evaluated by DLS and filter potency.
DLS (Diameter): 40% DL with Sodium glycohlate: 68.1±25 nm. 40% DL with SLS: 87.1±2.9 nm

### Filter Potency:

TABLE 1
Fraction of grazoprevir that passed through various sized filters for
four different grazoprevir/Ethylcellulose nanoparticle suspensions.

| | 40% DL | | 60% DL | |
|---|---|---|---|---|
| | with | 40% DL | with | 60% DL |
| | sodium | with | sodium | with |
| Condition | glycholate | SLS | glycholate | SLS |
| As is | 100 | 100 | 100 | 100 |
| 1 μm filtered | 100.7 | 98.9 | 96.9 | 99.4 |
| 0.45 μm filtered | 96.4 | 95.5 | 91.5 | 78.2 |
| 0.2 μm filtered | 82 | 92 | 29.3 | 23.3 |

60% Drug Load Nanoparticle: grazoprevir (60 mg) and ethylcellulose (40 mg) were dissolved into 6 mL dichloromethane until all components were fully dissolved. The 40% DL procedure listed above was then followed.
DLS (Diameter): 60% DL with sodium glycholate: 106.9±2 nm. 60% DL with SLS: 117.7±13.6 nm

### In Vivo Dog Study

Two nanosuspension formulations (33.3% DL grazoprevir/66.7% DL ethylcellulose and 66.7% DL grazoprevir/33.3% ethylcellulose) were prepared in lmg/mL SLS. A benchmark formulation of the FMF SDI was made by suspending the SDI in SGF and mixing to yield a uniform suspension. The suspension API concentration was 4 mg/mL.

Six male beagle dogs were used in the study. All dogs were fasted overnight prior to dosing and through the first 4 hours of blood sample collection. All dogs were pretreated with pentagastrin solution at a dose of 0.006 mg/kg. Water was removed/withheld beginning at the time of dosing and returned after completion of the 1 hr sample collection.

The study was designed in a cross over fashion between the two nanoformulations and the benchmark FMF SDI formulation. Each dog was dosed orally 24 mL of one of the test formulations followed by rinising with 10 mL water for nanosuspension or 10 mL SGF for the FMF SDI suspension.

Blood samples (ca. 0.5 mL) were collected from the jugular vein into tubes containing potassium EDTA as an anti-coagulant pre-dose and at the following sampling times: 0.25, 0.5, 1, 2, 4, 6, 8 and 24hr post dose. The plasma was seperated by centrifugation and frozen at −70° C. for storage and transferring until analysis. There were no adverse signs observed in any animals and all dose administrations were performed without incident.

Plasma samples were analyzed using a validated LC/MS method.

Area under the curve (AUC_{0-24 hr}), observed maximum plasma concentration (Cₘₐₓ) and time of Cₘₐₓ (Tₘₐₓ) were calculated using a linear trapezoidal, non-compartmental modle of WinNonlin® v 5.3. Plasma profiles were generated using WinNonLin in V5.3 or Microsoft® Excel 2010. Concentrations below lower limit of quantitation (LLOQ) were set to zero for calculation purposes.

TABLE 2
Mean pharmacokinetic parameters of grazoprevir dosed as a 66%
or 33% DL ethylcellulose nanoparticle suspension (in 0.1% SLS)
or a FMF suspension (in 0.1N HCl) in male beagle dogs (n = 6).

| | Dose | AUC 0-24 hr | | | AUC vs |
|---|---|---|---|---|---|
| Formulation | (mg) | (uMhr) | Cmax (uM) | Tmax (hr) | FMF |
| 33% DL nano | 100 | 88.5 ± 14.9 | 11.1 ± 1.0 | 2 | 0.47 |
| 66% DL nano | 100 | 131 ± 26 | 13.9 ± 1.8 | 3 | 0.7 |
| FMF SDI | 100 | 187 ± 12 | 20.7 ± 1.6 | 2 | 1.00 |

40% DL Grazoprevir/60% Ethylcellulose nanoparticle: grazoprevir (20 mg) and ethylcellulose grade 4 (30 mg) were dissolved into 2 mL of methanol. Aliquots of this solution were added to stirred water or 1 mg/mL SLS in water. Samples were characterized by DLS. DLS (diameter) in SLS: 154±34.5 nm. In water 157±21.5 nm.

### HPMCAS-H Based Nanoparticle Systems

Batch Precipiation of Grazoprevir/HPMCAS-H Nanoparticle Systems

An aqueous solution of pH=7.3 HPMCAS-H (1-10 mg/mL) was generated by titrating a suspension of HPMCAS-HF with 1N NaOH. grazoprevir was dissolved into acetone at 10-100 mg/mL. Suspensions were generated by adding the organic solution to a rapidly stirring aqueous solution.

Flow Precipiation of a Grazoprevir HPMCAS-H Nanoparticle System

A stock solution of grazoprevir (10 mg/mL) was prepared. A HPMCAS-H aqueous stock solution was prepared by suspending the polymer in water and titrating with 1N NaOH until polymer was dissolved and the final pH was between 7.3 and 7.4. The solutions mixed by either a T-cell mix or a mutli-inlet vortex mixer according to the chart below. The resulting suspensions were characterized by DLS and HPLC filter potency.

TABLE 3
Table of various flow precipiation schemes attempted for
50% DL grazoprevir/HPMCAS-H nanoparticle suspsensions.

| | | Flow Organic | Flow Aqueous |
|---|---|---|---|
| Trial | Cell | (mL/min) | (mL/min) |
| 1 | MIVM | 8 | 24 × 3 |
| 2 | Tcell (0.15) | 1 | 9 |
| 3 | MIVM | 4 | 12 × 3 |
| 4 | T cell (0.5) | 1 | 9 |
| 5 | T cell (0.15) | 5 | 45 |
| 6 | T cell (0.15) | 3 | 27 |

TABLE 4
Particle size measured by DLS and percent of grazoprevir
and HPMCAS-H passed through a 0.45 μm filter.

| | Zavg diameter | % Passed | % Passed |
|---|---|---|---|
| Trial | (nm) | grazoprevir | HPMCAS-H |
| 1 | 90.9 ± 1.1 | 92.12 | 103.8 |
| 2 | 94.4 ± 17.4 | 98.56 | 96.1 |
| 3 | 147.3 ± 1.3 | 92.85 | 103.3 |
| 4 | 112.8 ± 0.7 | 91.48 | 94.9 |
| 5 | Micron | | |
| 6 | Micron | | |

### Spray Drying of Grazoprevir/HPMCAS-H Nanoparticle Suspension.

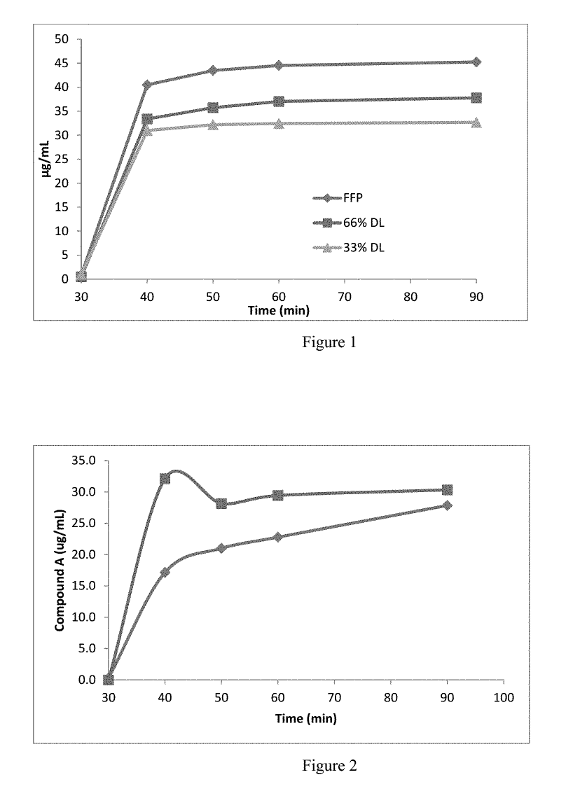

Grazoprevir nanoparticle suspensions were spray dried on a Procept Formamatrix lab scale spray dryer. The drying gas was air run at 0.45 m³/min. The outlet temperature of the system was set between 55-60° C. and a bifluid nozzle (0.6mm diameter) was set on the system. Nozzle atomization air was set to 9 L/min. Fluid spray rate was set to 2 mL/min. The resulting powder was characterized by DLS and filter potency after redispersion into water. DLS: 221±36.6 nm. % Passed 0.45 μm filter: 79.7±4.2%. FIG. 2 shows dissolution of two different 50% DL grazoprevir/HPMCAS-H nanoformulations as a nanosuspension (squares) and after spray drying (diamonds). Both formulations achieved ˜25 μg/mL solubility in Fassif by the end of the experiment, with the suspension formulation achieving this value by the first datapoint.

### HPMCAS-L Based Nanoparticle Systems

Small scale batch precipitation: Aqueous solutions of HPMCAS-L were created by suspending HPMCAS-L (5 mg/mL) in water and titrating with 1N sodium hydroxide to create a dissolve the polymer at a target pH of 5.5 or 7.5. An organic solution of grazoprevir was created at 35 mg/mL in acetone. The organic solution (143 μL) and aqueous solution (1 mL) were combined in a 1.5 mL centrifuge tube and vortexed for 30 seconds The resulting dispersion were analyzed by DLS.

TABLE 5
DLS data of grazoprevir/HPMCAS-L nanos prepared
under two different batch conditions.

| Initial pH | Diameter(nm) |
|---|---|
| 5.5 | 86.1 ± 19.1 |
| 7.5 | 107.1 ± 25 |

### Flow Precipiation:

A stock solution of grazoprevir (50 mg/mL) was created in acetone. HPMCAS-L (5.55 mg/mL) was suspended into water and the pH was adjusted to 7.5 using 1N NaOH and/or 1 N HCl. Nanoparticles were precipitated on a 0.5 mm ID T-mixer at a 9/1 aqueous to organic flow ratio with a 100 mL/min total flow rate. The resulting suspension was characterized by DLS and filter potency. DLS: 207.1±9.9 nm. %

### Spray Drying of Compound A/HPMCAS-Lnanoparticle Suspension.

Grazoprevir/HPMCAS-L nanoparticle suspensions were spray dried on a Procept Formamatrix lab scale spray dryer. The drying gas was air run at 0.45 m³/min. The outlet temperature of the system was set between 55-60° C. and a bifluid nozzle (0.6 mm diameter) was set on the system. Nozzle atomization air was set to 9 L/min. Fluid spray rate was set to 2 mL/min. The resulting powder was characterized by DLS, filter potency, PXRD, accelerated stability and dissolution.
DLS: 211 nm. Tg: 156° C. Amorphous by PXRD.

TABLE 6
Filter potency data of 50% DL grazoprevir/50% HPMCAS-L
nanoparticle formulation as a newly created suspension
and after drying and reconstitution.

| Filter | Initial Suspension | Reconsituted SD material |
|---|---|---|
| 1 μm | 100 | 100 |
| 0.45 μm | 100 | 91 |
| 80K centrifugation | 4.2 | 3.1 |

TABLE 7
Determination of solution content of grazoprevir
and HPMCAS-L after 80k centrifugation

| | % Grazoprevir | % HPMCAS-L |
|---|---|---|
| Treatment | in solution | in solution |
| 80K centirfugation | 4.2 | 100 |

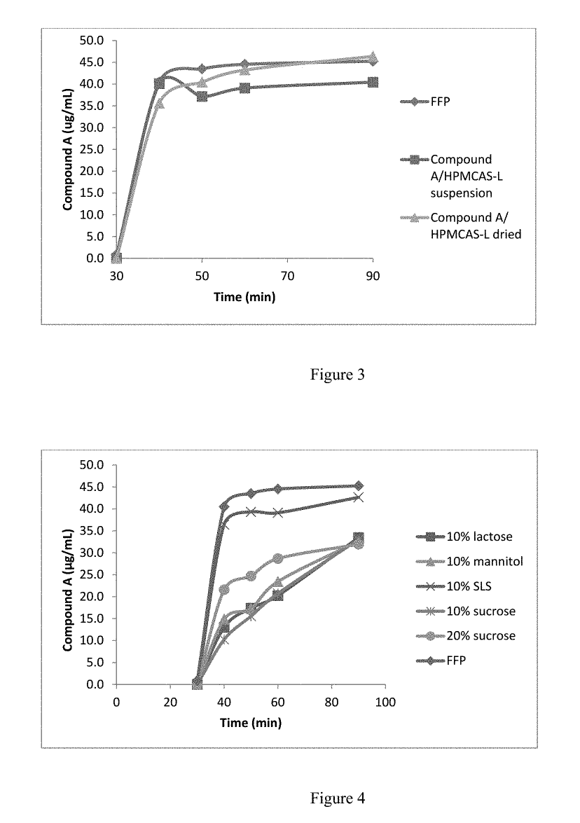

FIG. 3 describes the dissolution of 50% Grazoprevir/HPMCAS-L nanoparticle suspension (squares), the same formulation post-spray drying (triangle) compared to the commerically marketed formulation (diamonds). All three formulations had the same extent of release over time; however the nanosuspension and the commercially marketed formulation immediately achieved full release. FIG. 4 describes PXRD data of 50% Grazoprevir/HPMCAS-L nanoparticles after 4 weeks storage at 40° C./75% RH. The formulation remained amorphous over this period.

Formulation of Grazoprevir/HPMCAS-L nanoparticles with additives 50% DL Grazoprevir/HPMCAS-L nanoparticle suspension was generated as described above. The formed suspensions were split into equal aliquots and additional excipients were added to create the formulations described in the table below. The formulations were spray dried as described above.

TABLE 8
Particle size data for grazoprevir/HPMCAS-L formulations with
various addatives after spray drying and reconsitution.

| Formulation | Diameter (nm) |
|---|---|
| 45% Compound A/45% | 150.8 ± 2.5 |
| HPMCAS-L/10% SLS | |
| 45% Compound A/45% | 158.3 ± 1.3 |
| HPMCAS-L/10% lactose | |
| 40% Compound A/40% | 130.8 ± 2.0 |
| HPMCAS-L/ 20% sucrose | |
| 45% Compound A/45% | 151.8 ± 0.72 |
| HPMCAS-L/10% mannitol | |
| 45% Compound A/45% | 90.7 ± 3.9 |
| HPMCAS-L/105 sucrose | |

FIG. 4 describes the dissolution of 45% grazoprevir/45% HPMCAS-L/10% additive formulations. The SLS containing formulation was the only formulation able to match the dissolution behavior of the commerically marketed formulation.

### Optimization of Nanoparticle SLS Level

A stock solution of grazoprevir (50 mg/mL) was created in acetone. HPMCAS-L (5.55 mg/mL) was suspended into water and the pH was adjusted to 7.5 using 1N NaOH and/or 1 N HCl. A stock aqueous solution of SLS was added to make 0.1%, 1%, 2.5% or 5% total percentage of SLS. Nanoparticles were precipitated on a 0.5 mm ID T-mixer at a 9/1 aqueous to organic flow ratio with a 100 mL/min total flow rate. The resulting suspension was spray dried as described above and analyzed by dissolution.

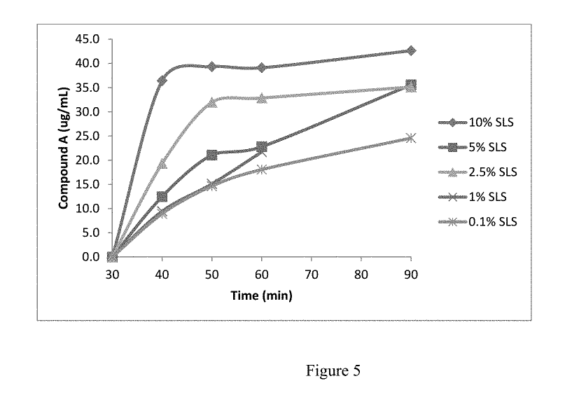

FIG. 5 describes the dissolution of grazoprevir/HPMCAS-L nanoformulations with various levels of SLS. 10% SLS was necessary to match the dissolution behavior of the commercially marketed formulation.

### Scale up of Grazoprevir/HPMCAS-L/SLS (45/45/10) Nanoparticle Suspension

Compound A was dissolved in acetone (50 mg/mL). HPMCAS-L and SLS (5.5 mg/mL and 0.55 mg/mL) were added to water and the pH was titrated to 7.5 with NaOH to dissolve the polymer. Nanoparticles were precipitated using a t-mix (0.5 mm inner diameter) at a 9/1 aqueous to organic flow ratio. The total flow rate was 75 ml/min. The resulting suspension was spray dried as described above and analyzed by laser diffraction and powder x-ray diffraction. Median diameter: 127 nm. X-ray amorphous.

### Encapsulation of Grazoprevir Nanoparticle Solids

In a small glass bottle (1 oz.), the intermediate (nanoparticle spray dried material, a spray dried intermediate of identical chemical composition or the FMF SDIS) was mixed with lactose and sodium chloride (<53 μm particle size) and passed through a #60 mesh screen (150 μm). The mixture was blended for 5 min on a Turbula blender. All other excipients (microcrystalline cellulose “Avicel 101”, sodium croscarmellose, and magnesium stearate) were added to the bottle, hand mixed and passed through a #60 mesh screen before blending. The mixture was blended for 5 minutes on a Turbula (46 RPM). The blended material of each formulation was slugged to four 2 g soft tablets on an MTS. The slugs milled forced through a #30 mesh screen (600 μm.) before 5 min blending. Each final blend was encapsulated in size 00 gelatin capsules by hand (400 mg for the nanoparticle formulation and its SD comparator and 453 mg for FMF SDI.) The materials were analyzed by dissolution.

TABLE 9
Composition of grazoprevir capsules evaluated in dog PK study.

| | grazoprevir/ | grazoprevir |
|---|---|---|
| | HPMCAS-L | (FMF) market |
| Ingredient | nanoformulation | comparator |
| 45% grazoprevir/45% | 27.95 | |
| HPMCAS-L/10% SLS | | |
| nanoparticle solid | | |
| formulation | | |
| grazoprevir Commercial | | 36.79 |
| Market Formulation | | |
| Microcrystalline Cellulose | 14.09 | 12.36 |
| (Avicel PH101) | | |
| Lactose, 316 Fast Flo | 42.26 | 37.08 |
| Sodium chloride | 10.06 | 8.83 |
| Croscarmellose sodium | 5.03 | 4.41 |
| Colloidal Silica | 0.1 | 0.1 |
| Magnesium stearate | 0.5 | 0.44 |
| Fill Weight (mg) | 397.5 | 453.03 |

### Dog PK of Grazoprevir Nanoformulations

Dog PK studies were performed at Charles River laboratories according to an approved IACUC protocol. Six male beagle dogs were fasted overnight and pretreated with pentagastrin 30 minutes prior to dosing via an intramuscular injection (0.006 mg/kg). Food was given 4 hrs post-dose. Water was removed before dosing until 1 hour post dose where it was available ad libitum for the remainder of the study. All six dogs were dosed with either grazoprevir nanoparticles in capsules, Compound A FMF SDI in capsules, followed by 3.5 mL/kg of water rinse.

Blood samples (ca 1 mL) were collected into tubes containing potassium EDTA as an anti-coagulant predose and at the following sample times (0.25, 0.5, 1, 2, 4, 6, 8 and 24hr post dose). Plasma was separated by centrifugation and kept frozen at −70° C. for storage and transferring until analysis. Plasma samples were analyzed using a validated grazoprevir LCMS method.

TABLE 10
PK parameters for Compound A formulations
dosed in beagle dogs.

| Formulation | AUC (h*nmol/L) | Cmax (nM) | Tmax (hr) |
|---|---|---|---|
| grazoprevir/ | 177912 ± 28748 | 27716 ± 3899 | 4 |
| HPMCAS-L | | | |
| nanoformulation | | | |
| FMF SDI capsule | 130010 ± 19488 | 21057 ± 1584 | 4 |
| formulation | | | |

### PVPVA64 Based Formulations

Small scale precipitation: The following nanoparticle systems were precipitated. Compound A was dissolved into 4 mL acetone and PVP-VA64 (obtained from BASF) and SLS were dissolved in 36 mL of water. The aqueous solutions were vigororusly stirred and the organic solution was added at the center of the stir vortex. Formulations were analyzed by particle size by laser diffraction and then spray dried via the parameters described previously. Each of the particles were added to a 100 mL bottle, targeting 5 mg API. Dissolution analysis was done as described above, with drug concentrations determined after either 1 um filtration (% nano) or 80 k centrifugation (dissolved drug)

TABLE 11
Particle size, % nano, % dissolved and Fassif solubility
for grazoprevir/PVPVA-64 nanoformulations.

| | | | % | % | |
|---|---|---|---|---|---|
| | Particle | % | Nano | Dissolved | Fassif |
| | Diameter | Nano | in | in | Soly |
| Compostion | (nm) | in SGF | Fassif | Fassif | (ug/mL) |
| 40% DL, 60% | 125 | 1.85 | 86.2 | 72.5 | 40.2 |
| PVPVa64 | | | | | |
| 40% DL/50% | 87 | 11.0 | 78.14 | 68.28 | 42.8 |
| PVPVA64/ | | | | | |
| 10% SLS | | | | | |
| 66% DL/33% | 145 | <0.25 | 90.1 | 67.2 | 24.3 |
| VA64 | | | | | |
| 66% DL/23% | 81 | 0.31 | 79.9 | 67.1 | 25.4 |
| PVPVA64/ | | | | | |
| 10% SLS | | | | | |
| 50% DL/50% | 97 | 1.04 | 90.91 | 70.52 | 32.9 |
| PVPVA64 | | | | | |
| 50% DL/45% | 84 | 72 | 81.9 | 65 | 38.8 |
| VA64/5% SLS | | | | | |

What is claimed is:

1. A polymer-stabilized nanoparticle comprising from 0.5 to 15 weight percent of anionic surface modifying agent selected from the group consisting of sodium lauryl sulfate, sodium glyocholate, and sodium taurocholate, or a mixture thereof, from 20 to 80 weight percent of a water soluble polymer or poorly aqueous soluble polymer and from 20 to 80 weight percent of a compound that is grazoprevir represented by formula I: 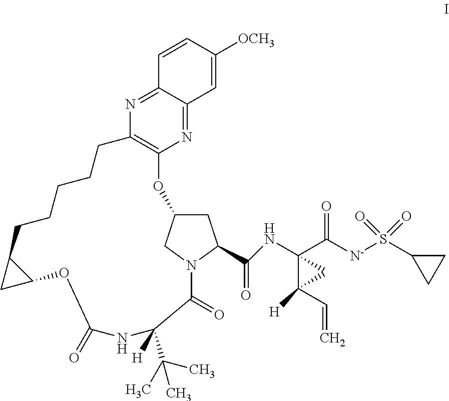 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof.

2. The polymer-stabilized nanoparticle of claim 1, comprising about 8 to about 10 weight percent of an anionic surface modifying agent, from about 30 to about 65 weight percent of a water soluble polymer or poorly aqueous soluble polymer and from about 30 to about 65 weight percent of grazoprevir.

3. The polymer-stabilized nanoparticle of claim 1, wherein the anionic surface modifying agent is sodium lauryl sulfate.

4. The polymer-stabilized nanoparticle of claim 1, wherein anionic surface modifying agent is from 10 to 25% on a weight basis to grazoprevir.

5. The polymer-stabilized nanoparticle of claim 4, wherein anionic surface modifier modifying agent is from 20 to 24% on a weight basis to grazoprevir.

6. The polymer-stabilized nanoparticle of claim 1, wherein, the poorly aqueous soluble polymer is selected from the group consisting of hydroxypropylmethylcellulose (HPMCAS), ethylcellulose (EC), propylcellulose, and butylcellulose.

7. The polymer-stabilized nanoparticle of claim 1, wherein the anionic surface modifying agent is sodium lauryl sulfate, from 20 to 24% on a weight basis to grazoprevir.

8. The polymer-stabilized nanoparticle of claim 1 wherein, the poorly aqueous soluble polymer is an enteric polymer having a low aqueous solubility at pH levels below 6.

9. The polymer-stabilized nanoparticle claim 8 wherein the poorly aqueous soluble polymer is hydroxypropylmethylcellulose (HPMCAS)

10. The polymer-stabilized nanoparticle of claim 1, wherein the water soluble polymer is selected from the group consisting of polyvinylpyrollidone-vinyl alcohol, polyvinyl pyrollidinone, polyethylene glycol, and polyvinyl alcohol, or a mixture thereof.

11. The polymer-stabilized nanoparticle of claim 100 wherein the water soluble polymer is polyvinylpyrollidone-vinyl alcohol.

12. The polymer-stabilized nanoparticle of claim 1 which also comprises from 0.1-10% of an additive.

13. The polymer-stabilized nanoparticle of claim 12 wherein the additive is selected from the group consisting of sucrose, lactose, mannitol, casein, trehalose, cellulose and mesoporous silica, or a mixture thereof.

14. The polymer-stabilized nanoparticle of claim 1 wherein the particle size is between 5-20 μm with a drug load of 45% to 70%.

15. A polymer-stabilized nanoparticle comprising a polymer ratio range from 30-65% of water soluble polymer or poorly aqueous soluble polymer, 5-10% of sodium lauryl sulfate, and 33-66% of the active agent a compound that is grazoprevir represented by formula I: 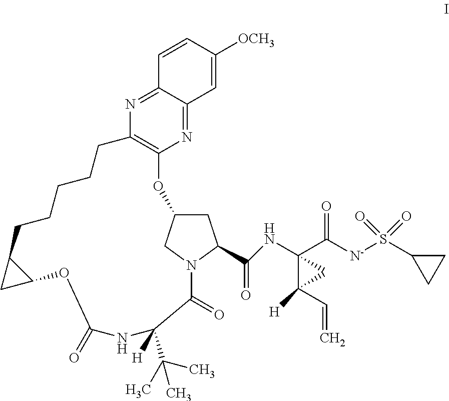 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof

16. The polymer-stabilized nanoparticle of claim 15 which also comprises 0.1-10% of an additive selected from the group consisting of sucrose, lactose, mannitol, casein, trehalose, cellulose and mesoporous silica, or a mixture thereof.

17. The polymer-stabilized nanoparticle of claim 15 wherein the particle size is between 5-20 μm with a drug load of 45% to 70%.

18. A polymer-stabilized nanoparticle of claim 1 wherein the nanoparticles comprise 100 to 500 nm of amorphous grazoprevir, or a pharmaceutically acceptable salt thereof.

19. A polymer-stabilized nanoparticle of claim 1 wherein the nanoparticle releases from 40 to 100% of the grazoprevir when placed in solution at a temperature of 35° C. to 37° C.

20. A polymer-stabilized nanoparticle of claim 1 wherein the nanoparticle releases from 40 to 100% of the grazoprevir when placed in intestinal fluid at a temperature of 35° C. to 37° C.

21. A pharmaceutically acceptable composition comprising a plurality of polymeric nanoparticles of claim 1 and a pharmaceutically acceptable excipient.

22. A method of treating hepatitis C virus in a patient in need thereof, comprising administering to the patient a therapeutically effective amount of a composition comprising the polymeric nanoparticle of claim 1.

23. A process for making stabilized polymeric nanoparticles having a polymer ratio range from a 30-65% of water soluble polymer or poorly aqueous soluble polymer, sodium lauryl sulfate ratio from 5-10%, and a ratio from 33-66% of grazoprevir represented by formula I: 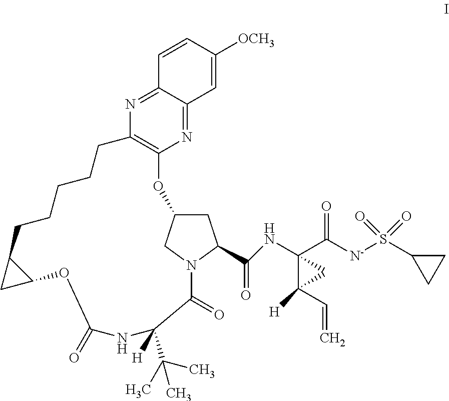 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof comprising the steps of forming an organic solution comprising an organic solvent and the active agent, forming an aqueous solution comprising dissolved polymer and surface modifying agent, mixing said organic and aqueous solutions together to form nanosuspensions, removing said solvents from the suspension to form a heterogeneous micron sized particle of said nanoparticles embedded within the polymer and surface modifying agent and collecting said nanoparticles.

24. The process according to claim 23 wherein the organic solvent is miscible with the aqueous solution.

25. The process according to claim 23 wherein the solvent is selected from the group consisting of acetone, methanol, ethanol, tetrahydrofuran (THF), dimethylsulfoxide (DMSO), or any mixture thereof.

26. The process according to claim 23 which yields from about 100-200 nm particles of pure amorphous grazoprevir that are stabilized by polymeric excipients.

27. The process according to claim 23 wherein grazoprevir is optionally ionized by pH modifying agents selected from sodium hydroxide, potassium hydroxide, ammonia, sodium phosphate, lysine and arginine.

28. The process according to claim 23 which yields a particle size between 5-20 μm with a drug load of 45% to 70%.
